(19) United States
(12) Patent Application Publication
(10) Pub. No.: US 2024/0350215 A1
(43) Pub. Date: Oct. 24, 2024

(54) UNIVERSAL SURGICAL ROBOTIC PLATFORM

(71) Applicant: Andromeda Surgical, Inc., San Francisco, CA (US)

(72) Inventors: Nicholas Ralph Damiano, San Francisco, CA (US); Kartik Tiwari, San Jose, CA (US)

(73) Assignee: Andromeda Surgical, Inc., San Francisco, CA (US)

(21) Appl. No.: 18/640,967

(22) Filed: Apr. 19, 2024

Related U.S. Application Data
(60) Provisional application No. 63/460,698, filed on Apr. 20, 2023.
(60) Provisional application No. 63/536,651, filed on Sep. 5, 2023.

(51) Int. Cl. A61B 34/37; A61B 34/00; A61B 34/30; A61B 90/00

(52) U.S. Cl. CPC A61B 34/37; A61B 34/25; A61B 90/361; A61B 2034/304

(57) ABSTRACT

A robotic system includes a processor and a robotic platform in communication with the processor. The robotic platform is configured to couple to a first procedure-specific end effector. The first procedure-specific end effector is controllable by the robotic platform and is selected to perform a first type of procedure. The processor is configured to receive first procedure-specific code and to execute the first procedure-specific code to cause the robotic platform to operate the first procedure-specific end effector to perform the first type of procedure.

### CROSS-REFERENCE TO RELATED APPLICATIONS

This application claims the benefit of and priority to U.S. Provisional Patent Application No. 63/460,698, filed Apr. 20, 2023, and U.S. Provisional Patent Application No. 63/536,651, filed Sep. 5, 2023, the contents of each of which are incorporated herein by reference in their entirety.

### FIELD

This disclosure relates to universal surgical robotic platforms and methods of using the same.

### BACKGROUND

Traditionally, surgeries performed by human clinicians are limited by human senses, physical abilities, and judgment. Such limitations can reduce the efficacy of surgeries performed by human clinicians. In recent years, to address such shortcomings, surgical robots have been developed that can increase the speed, efficiency, and precision of surgeries.

### SUMMARY

The present disclosure relates to a robotic system, including: a processor; and a robotic platform in communication with the processor, wherein: the robotic platform is configured to couple to a first procedure-specific end effector, the first procedure-specific end effector controllable by the robotic platform and being selected to perform a first type of procedure; and the processor is configured to receive first procedure-specific code and to execute the first procedure-specific code to cause the robotic platform to operate the first procedure-specific end effector to perform the first type of procedure.

The present disclosure relates to a method, including: uploading a first procedure-specific code to a robotic platform, wherein the first procedure-specific code, when executed by a processor of the robotic platform, causes the robotic platform to operate a first procedure-specific end effector to perform a first type of procedure; attaching the first procedure-specific end effector to the robotic platform; and causing the robotic platform to perform the first type of procedure.

The present disclosure relates to a system for coupling an end effector to a robotic platform, including: an adapter configured to be coupled to a robotic arm of the robotic platform; and a customized holder couplable to the adapter, wherein the customized holder is configured to hold the end effector.

The present disclosure relates to an adapter, including: a body including: a first end couplable to a robotic arm; and a second end; and a cantilever extending distally from the second end of the body, wherein the cantilever includes a side face defining a mating surface of the adapter at which the adapter is configured to couple to a customized holder, wherein the mating surface includes: an electromagnet; and a plurality of protuberances surrounding the electromagnet and defining one or more recesses between the plurality of protuberances.

The present disclosure relates to a customized holder, including: a base defining a mating surface at which the customized holder is configured to couple to a mating surface of an adapter, wherein the mating surface includes: a magnetic metal; and a plurality of keys surrounding the magnetic metal; a plurality of cantilevers extending from the base; a locking member rotatably coupled to the plurality of cantilevers at a first end of the locking member; and a retainer configured to receive a second end of the locking member, wherein the customized holder is configured to hold an end effector in a space defined at least partially between the plurality of cantilevers and the locking member when the second end of the locking member is received by the retainer; the plurality of cantilevers are shaped and sized based on the end effector to be held in the space defined at least partially between the plurality of cantilevers and the locking member when the second end of the locking member is received by the retainer; and the locking member is shaped and sized based on the end effector to be held in the space defined at least partially between the plurality of cantilevers and the locking member when the second end of the locking member is received by the retainer.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. TA depicts a universal surgical robotic platform, according to one or more embodiments described herein;

FIG. 1B depicts a method of using a universal surgical robotic platform, according to one or more embodiments described herein;

FIG. 2 depicts a schematic representation of a universal surgical robotic platform, according to one or more embodiments described herein;

FIG. 3 depicts an exemplary graphical user interface, according to one or more embodiments described herein;

FIG. 4A depicts an exemplary end effector coupled to a robotic arm via an adapter and holder, according to one or more embodiments described herein;

FIG. 4B depicts an exploded view of FIG. 4A, according to one or more embodiments described herein;

FIG. 5 depicts an example holmium laser enucleation of the prostate procedure, according to one or more embodiments described herein; and

FIG. 6 depicts an example computing device, according to one or more embodiments described herein.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The efficacy of surgical procedures has traditionally been largely dependent on a particular surgeon's skill level and experience with a specific surgery. That is, a surgeon may need to conduct a certain number of surgeries of a particular kind (for instance holmium laser enucleation of the prostate (HoLEP)) before the surgeon becomes proficient in the surgery such that desirable and precise surgical outcomes can be expected with a high degree of certainty. Even then, however the outcome of surgical procedures has been limited by human error of the surgeon. Such errors can be in decision making and physical precision (e.g., physical manipulation of a surgical instrument). Human surgical error, in many cases, has been tied to shortcomings in the surgeon's ability to accurately visualize the surgical target area in the patient's body and the surgeon's ability to precisely control surgical instrumentation in the surgical site.

In recent years, to address such shortcomings, surgical robots have been developed that can increase the speed, efficiency, and precision of surgeries. However, the practicality of such surgical robots has been limited due to the need for specially trained and equipped robots for particular procedures. That is, a first robotic platform is designed and used for a first type of procedure, and a second robotic platform is designed and used for a second type of procedure.

The present disclosure aims to address the current shortcomings in the state of the art of surgical robots with a universal surgical robot platform that can be equipped and modified to perform different types of procedures. The universal surgical robotic platform can include a cart, or other platform, and one or more movable elements, such as a robotic arm. The universal surgical robotic platform can be modified with procedure-specific code and procedure-specific end effectors to carry out any desired procedure. That is, the platform and movable elements of the universal surgical robotic platform can remain unchanged from a first procedure to a second procedure, and new procedure-specific code and one or more procedure-specific end effectors can be uploaded or attached, respectively, to the universal surgical robotic platform to perform the second procedure. Such a modifiable universal surgical robotic platform can greatly reduce layover time between surgical procedures and reduce functional and economical burden of needing different robotic platforms to each perform unique procedures.

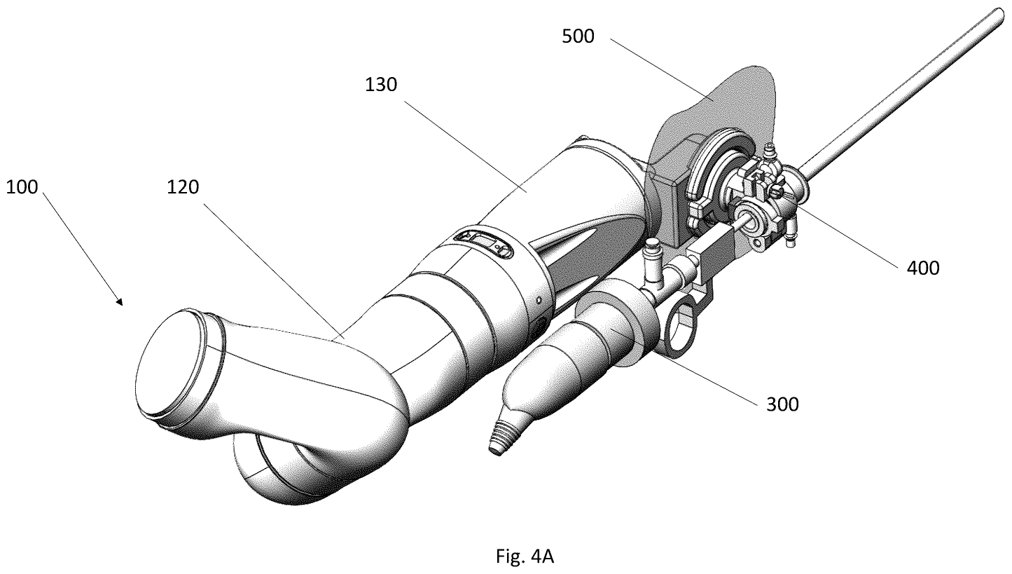

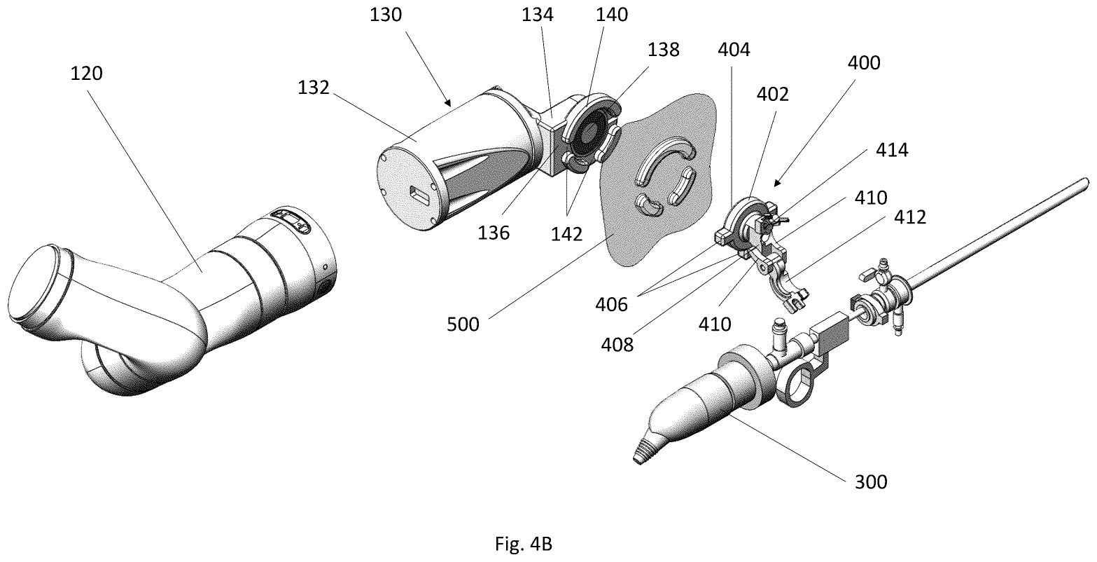

Referring to FIG. TA, a universal surgical robotic platform 100 is generally depicted. The universal surgical robotic platform 100 includes software and hardware components that can be trained or modified to perform various types of procedures. The universal surgical robotic platform 100 can include a software interface and a processor, or other controller, to receive inputs 200, including procedure-specific code 202 and other data 204, such as imaging data or kinematic data. The universal surgical platform 100 can include one or more moveable robotic arms. “Robotic arms,” as the term is used herein, can refer to any mechanically movable element of the universal surgical robotic platform 100 that can manipulate an end effector. In some embodiments, the “robotic arm” referred to herein can include multiple links coupled together by one or more joints. In some embodiments, the “robotic arm” referred to herein can include a single, non-jointed link. In some embodiments, “robotic arm” can refer to a flexible element coupled to the universal surgical robotic platform 100. The universal surgical platform 100 can include a hardware interface, such as an adapter, configured to receive one or more procedure-specific end effectors 300, as discussed further below with respect to FIG. 4A and FIG. 4B. The hardware interface can allow the one or more robotic arms to manipulate the one or more procedure-specific end effectors 300, can allow for non-permanent coupling of the one or more robotic arms or other portion of the universal surgical platform 100 with the one or more procedure-specific end effectors 300, and the like. For instance, in some embodiments, the hardware interface can be coupled to the robotic arm and the one or more procedure-specific end effectors 300.

The universal surgical robotic platform 100 can autonomously or semi-autonomously perform various types of procedures. Thus, “performing” a procedure, as discussed with respect to the universal surgical robotic platform 100 or any components controlled, actuated, or moved by the universal surgical robotic platform 100 herein, can mean performing the procedure in its entirety, performing the procedure partially, or performing one or more actions in the procedure to assist a surgeon. In some embodiments, the universal surgical robotic platform 100 is modular and thus can be modified based on a desired procedure. In some embodiments, the universal surgical robotic platform 100 can include the necessary processing components to receive procedure-specific code 202, such as algorithms, and execute the procedure-specific code 202 (i.e., control one or more hardware components in accordance with the instructions in the procedure-specific code, as discussed below). In some embodiments, the universal surgical robotic platform 100 can be pre-programmed with various procedure-specific codes 202. That is, the universal surgical robotic platform 100 can be pre-programmed with procedure-specific code 202 for executing a first type of procedure, procedure-specific code 202 for executing a second type of procedure, and so on. In some embodiments, a user can pre-operatively upload procedure-specific code 202 to the universal surgical robotic platform 100. In some embodiments, a user can train the universal surgical robotic platform 100 on procedure-specific code 202 as the user determines to use the universal surgical robotic platform 100 for a specific type of procedure. Specifically, in some embodiments, the user can pre-operatively upload procedure-specific code 202 for executing a first type of procedure prior to using the universal surgical robotic platform 100 to perform the first type of procedure, and the user can pre-operatively upload procedure-specific code 202 for executing a second type of procedure prior to using the universal surgical robotic platform 100 to perform the second type of procedure.

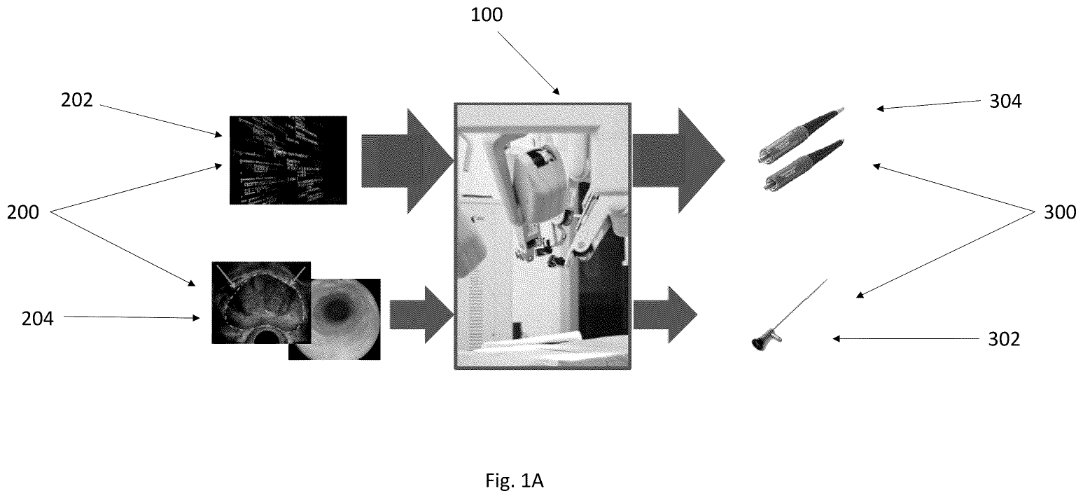

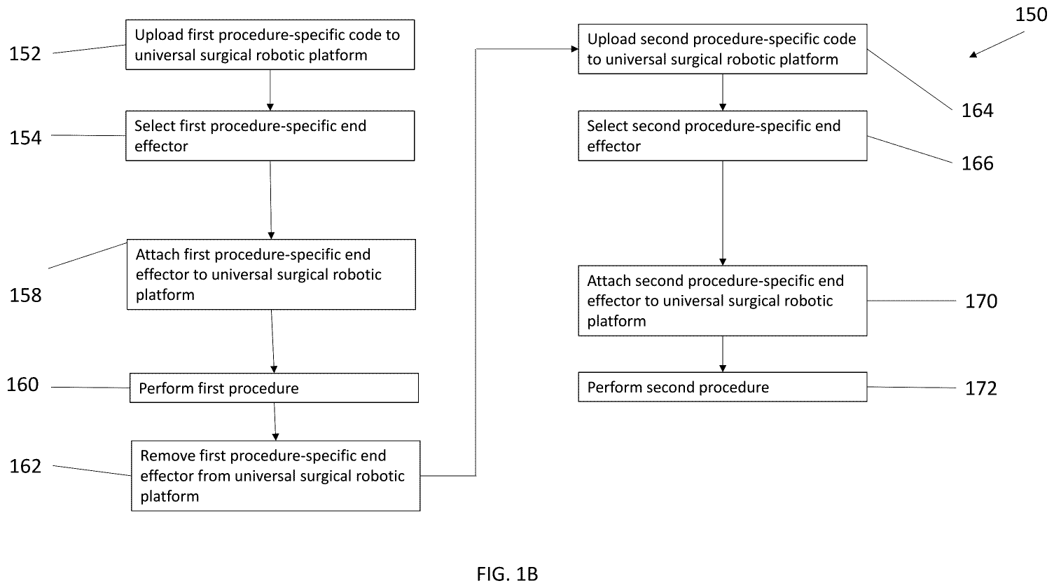

Still referring to FIG. 1A, the universal surgical robotic platform 100 can be configured to receive other data 204. In some embodiments, merely as an example, the other data 204 can be real-time imaging data from one or more imaging devices, such as ultrasound imaging devices, endoscopic imaging device, fluoroscopy imaging devices, MRI/ultrasound fusion imaging devices, MRI devices, CT devices, etc. In some embodiments, the imaging devices can be separate from the universal surgical robotic platform 100. In some embodiments, the imaging devices can be part of the universal surgical robotic platform 100. That is, the imaging devices can be included in the hardware of the universal surgical robotic platform 100, and the universal surgical robotic platform 100 can control the operation of the imaging devices. In some embodiments, the one or more imaging devices can be included in a procedure-specific end effector 300 coupled to the universal surgical platform 100 via the hardware interface. The universal surgical robotic platform 100 can include the necessary processing components to receive imaging data from the imaging devices, analyze the imaging data, and execute one or more operations or procedures based on the imaging data. That is, for instance, the universal surgical robotic platform 100 can receive and analyze the real-time imaging data during execution of a surgical procedure and use the real-time imaging data to guide the operation of the universal surgical robotic platform 100 and the procedure-specific end effector 300 coupled to the universal surgical robotic platform 100 (i.e., execute the procedure). It should be appreciated that imaging data is discussed merely as an example of other data 204, and that kinematic data or other feedback data can also be acquired as other data 204. The universal surgical robotic platform 100 can receive and analyze the other data 204, of any kind, during execution of surgical procedure and use the other data 204 to guide the operation of the universal surgical robotic platform 100 and the procedure-specific end effector 300 coupled to the universal surgical robotic platform 100 (i.e., execute the procedure).

Referring now to FIG. 1A and FIG. 4A, the universal surgical robotic platform 100 includes customizable hardware components for performing various types of procedures. For instance, the universal surgical robotic platform 100 can include one or more robotic arms 120 that are movable via one or more motor controllers and provide any desirable range of motion or degrees of freedom. The robotic arms 120 can be couplable to one or more procedure-specific end effectors 300 in order to perform various procedures. For instance, the distal ends of the robotic arms 120 can be coupled to first procedure-specific end effectors 300 to perform a first type of procedure, the distal ends of the robotic arms can be coupled to second procedure-specific end effectors 300 to perform a second type of procedure, etc. In some embodiments, the procedure-specific end effectors 300 can be gripped, held, or otherwise coupled to the distal ends of the one or more robotic arms 120. In some embodiments, the procedure-specific end effectors 300 can be non-fixedly coupled to the distal ends of the one or more robotic arms 120. In some embodiments, the procedure-specific end effectors 300 can be directly coupled to the one or more robotic arms 120, such that the one or more robotic arms 120 define the hardware interface for coupling the procedure-specific end effectors 300 to the universal surgical robotic platform 100.

In some embodiments, the universal surgical robotic platform 100 includes an adapter 130. In some embodiments, the adapter 130 can be coupled to the distal end of the robotic arm 120. In some embodiments, the procedure-specific end effectors 300 can be gripped, held, or otherwise coupled to the adapter 130. In some embodiments, the procedure-specific end effector 300 can be gripped, held, or otherwise coupled to the adapter 130 at or near the distal end of the adapter 130. In some embodiments, the procedure-specific end effectors 300 can be non-fixedly coupled to the adapter 130. In some embodiments, the adapter 130 can define the hardware interface for coupling the procedure-specific end effectors 300 to the universal surgical robotic platform 100. In some embodiments, the procedure-specific end effectors 300 can be directly coupled to the adapter 130.

In some embodiments, the procedure-specific end effectors 300 can be gripped, held, or otherwise coupled to one or more intermediary components, which are themselves gripped, held, or otherwise coupled to the universal surgical robotic platform 100. The intermediary component can be a customized holder 400. In some embodiments, the customized holder 400 can be a clamp. In some embodiments, the one or more intermediary components can be gripped, held, or otherwise coupled to the robotic arm 120 of the universal surgical robotic platform 100. In some embodiments, the one or more intermediary components can be gripped, held, or otherwise coupled to the adapter 130 of the universal surgical robotic platform 100. In some embodiments, the intermediary component can be gripped, held, or otherwise coupled to the adapter 130 at or near the distal end of the adapter 130, or be gripped, held, or otherwise coupled to the robotic arm 120 at or near the distal end of the robotic arm 120. In some embodiments, the intermediary component can be non-fixedly coupled to the adapter 130 or the robotic arm 120.

In some embodiments, the procedure-specific end effectors 300 can be gripped, held, or otherwise coupled to the one or more intermediary components, or customized holder 400. In some embodiments, the procedure-specific end effectors 300 can be non-fixedly coupled to the one or more intermediary components. In some embodiments, the procedure-specific end effectors 300 can be directly coupled to the intermediary components. In some embodiments, as discussed in further detail below, the one or more intermediary components can be a customized holder 400 physically designed for a particular procedure-specific end effector 300 to be coupled to the one or more intermediary components. Therefore, in some embodiments, a user can select a particular procedure-specific end effector 300 for coupling to the universal surgical robotic platform 100 in order to perform a desired procedure with the procedure-specific end effector 300, and the user can further select a particular intermediary component, or customized holder 400, based on the selected procedure-specific end effector 300, for coupling the selected procedure-specific end effector 300 to the universal surgical robotic platform 100. The procedure-specific end effector 300, alone or together with the intermediary components, can be described as a distal assembly herein. In some embodiments, the distal assembly can be separated from the universal surgical robotic platform 100 by a sterile drape 500.

Still referring to FIGS. 1A and 4A, in some embodiments, the procedure-specific end effectors 300 can be multi-purpose tools 302. The multi-purpose tools 302 can be used to perform more than one type of procedure. Merely as examples, a multi-purpose tool 302 can include an aspirator, a rigid or flexible endoscope, a biopsy needle system (for example, a prostate biopsy system), a transrectal, transvaginal, or transurethral ultrasound probe, or a transcatheter delivery system for a cardiovascular procedure. The multi-purpose tool 302 is a procedure-specific end effector 300 in that a user purposely selects the multi-purpose tool 302 and couples the multi-purpose tool 302 to the one or more intermediary components, the one or more robotic arms 120, the adapter 130, or otherwise attaches the multi-purpose tool 302 to the universal surgical robotic platform 100 to perform a desired procedure. In some embodiments, the procedure-specific end effectors 300 can be single-purpose tools 304 that can only be used to perform one type of procedure. In some embodiments, the procedure-specific end effectors 300 can be particularly designed or customized for use with the universal surgical robotic platform 100. Such procedure-specific end effector 300 can be modified to pair or couple specifically with the universal surgical robotic platform 100, the one or more robotic arms 120, the adapter 130, or the one or more intermediary components.

In some embodiments, the procedure-specific end effectors 300 may plug in directly into the universal surgical robotic platform 100. For example, the procedure-specific end effector 300 may be a flexible visualization device (for example, cystoscope) or a flexible robotic that may plug in directly to an interface on the universal surgical robotic platform 100, and the internal mechanisms of the universal surgical robotic platform 100 can move and control the procedure-specific end effectors 300 directly, without the use of additional robotic arms. In some embodiments, the procedure-specific end effectors 300 may plug in directly into the universal surgical robotic platform 100 for power supply and actuation of the procedure-specific end effector 300.

Referring now to FIG. 1B, a method 150 of operating the universal surgical robotic platform 100 is depicted. In step 152, a user can upload first procedure-specific code 202 to the universal surgical robotic platform 100. The first procedure-specific code 202 can enable the universal surgical robotic platform 100 to be used to perform a first procedure. The universal surgical robotic platform 100 can be used to perform the first procedure autonomously or semi-autonomously with varying degrees of user control and supervision. The first procedure-specific code 202 can enable a processor of the universal surgical robotic platform 100 to control a first procedure-specific end effector 300 for performing the first procedure. For instance, the first procedure-specific code 202 can inform the processor of the dimensions, rigidity, and operability (ablation vs. aspiration, ablation temperature, etc.) of the first procedure-specific end effector 300. The first procedure-specific code 202 can include algorithms for the processor to determine the position of the first procedure-specific end effector 300 relative the adapter 130 with kinematic feedback other data 204, for instance. In some embodiments, the first procedure-specific code 202 can include algorithms for the processor to determine the progress of the first procedure based on imaging data (e.g., other data 204) or feedback data on the position and actuation of the first procedure-specific end effector 300. In some embodiments, the first procedure-specific code 202 can include algorithms for the processor to identify relevant anatomical landmarks to the first procedure in imaging data received by the processor, and to label the relevant anatomical landmarks in the imaging data for presentation to a user. It should be appreciated that these are merely examples, and the first procedure-specific code 202 can include algorithms for the processor to make any procedure-specific determinations, controls, or presentations, such as without limitation, presenting procedure-specific alerts or alarms.

In step 154, of the method, the user can select a first procedure-specific end effector 300 to couple to the universal surgical robotic platform 100 for performing the first procedure. Examples of procedure-specific end effectors 300 are discussed in greater detail below. Generally, the procedure-specific end effector 300 can be a device with a desired operation (imaging, aspiration, irrigation, ablation, etc.) for performing the first procedure.

In step 158, the user can attach the first procedure-specific end effector 300 to the universal surgical robotic platform 100. In some embodiments, to attach the first procedure-specific end effector 300 to the universal surgical robotic platform 100, a user can select a first customized holder 400 for coupling the first procedure-specific end effector 300 to the universal surgical robotic platform 100. As discussed in further detail below, the first customized holder 400 can be an end effector-specific holder. That is, the first customized holder 400 can be specifically sized for receiving and retaining the first procedure-specific end effector 300 in the first customized holder 400. In some embodiments, the user can first couple the first procedure-specific end effector 300 with the first customized holder 400 (together a “distal assembly”), and then couple the first customized holder 400 to the universal surgical robotic platform 100. In some embodiments, the user can first couple the first customized holder 400 to the universal surgical robotic platform 100, and then couple to the first procedure-specific end effector 300 to the first customized holder 400. In some embodiments, the first customized holder 400 is coupled to the robotic arm 120. In some embodiments, the first customized holder 400 is coupled to the adapter 130 coupled to the robotic arm 120.

In step 160, the universal surgical robotic platform 100 can be used to carry out the first procedure. In some embodiments, the universal surgical robotic platform 100 can execute the first procedure-specific code 202 to autonomously perform the first procedure. In some embodiments, the universal surgical robotic platform 100 can receive user input and execute the first procedure-specific code 202 to semi-autonomously perform the first procedure (for instance, based on user instructions for guiding movement of the first procedure-specific end effector 300, instructions for actuating the first procedure-specific end effector 300, etc.).

In step 162, following completion of the first procedure, the user can remove the first procedure-specific end effector 300 from the universal surgical robotic platform 100. In some embodiments, the user can remove the first customized holder 400 and the first procedure-specific end effector 300 from the universal surgical robotic platform 100. In some embodiments, the user can remove the first customized holder 400 from the universal surgical robotic platform 100 (e.g., the adapter 130) while the first procedure-specific end effector 300 remains coupled to the first customized holder 400.

In step 164, a user can upload second procedure-specific code 202 to the universal surgical robotic platform 100. The second procedure-specific code 202 can enable the universal surgical robotic platform 100 to be used to perform a second procedure. The universal surgical robotic platform 100 can be used to perform the second procedure autonomously or semi-autonomously with varying degrees of user control and supervision. The second procedure-specific code 202 can enable a processor of the universal surgical robotic platform 100 to control a second procedure-specific end effector 300 for performing the second procedure. For instance, the second procedure-specific code 202 can inform the processor of the dimensions, rigidity, and operability (ablation vs. aspiration, ablation temperature, etc.) of the second procedure-specific end effector 300. The second procedure-specific code 202 can include algorithms for the processor to determine the position of the second procedure-specific end effector 300 relative the adapter 130 with kinematic feedback other data 204, for instance. In some embodiments, the second procedure-specific code 202 can include algorithms for the processor to determine the progress of the second procedure based on imaging data (e.g., other data 204) or feedback data on the position and actuation of the second procedure-specific end effector 300. In some embodiments, the second procedure-specific code 202 can include algorithms for the processor to identify relevant anatomical landmarks to the second procedure in imaging data received by the processor, and to label the relevant anatomical landmarks in the imaging data for presentation to a user. It should be appreciated that these are merely examples, and the second procedure-specific code 202 can include algorithms for the processor to make any procedure-specific determinations, controls, or presentations, such as without limitation, presenting procedure-specific alerts or alarms.

In step 166, the user can select a second procedure-specific end effector 300 to couple to the universal surgical robotic platform 100 for performing the second procedure. Examples of procedure-specific end effectors 300 are discussed in greater detail below. Generally, the procedure-specific end effector 300 can be a device with a desired operation (imaging, aspiration, irrigation, ablation, etc.) for performing the second procedure.

In step 170, the user can attach the second procedure-specific end effector 300 to the universal surgical robotic platform 100. In some embodiments, to attach the second procedure-specific end effector 300 to the universal surgical robotic platform 100, a user can select a second customized holder 400 for coupling the second procedure-specific end effector 300 to the universal surgical robotic platform 100. As discussed in further detail below, the second customized holder 400 can be an end effector-specific holder. That is, the second customized holder 400 can be specifically sized for receiving and retaining the second procedure-specific end effector 300 in the second customized holder 400. In some embodiments, the user can first couple the second procedure-specific end effector 300 with the second customized holder 400 (together a “distal assembly”), and then couple the second customized holder 400 to the universal surgical robotic platform 100. In some embodiments, the user can first couple the second customized holder 400 to the universal surgical robotic platform 100, and then couple the second procedure-specific end effector 300 to the second customized holder 400. In some embodiments, the second customized holder 400 is coupled to the robotic arm 120. In some embodiments, the second customized holder 400 is coupled to the adapter 130 coupled to the robotic arm 120.

In step 172, the universal surgical robotic platform 100 can be used to carry out the second procedure. In some embodiments, the universal surgical robotic platform 100 can execute the second procedure-specific code 202 to autonomously perform the second procedure. In some embodiments, the universal surgical robotic platform 100 can receive user input and execute the second procedure-specific code 202 to semi-autonomously perform the second procedure (for instance, based on user instructions for guiding movement of the second procedure-specific end effector 300, instructions for actuating the second procedure-specific end effector 300, etc.).

It should be appreciated that the order of operation discussed with respect to the method 150 is non-limiting. Several steps of the method 150 can be omitted, combined, or performed in a different operation. Merely as an example, a user can first select the first procedure-specific end effector 300 in step 154, and then upload the first procedure-specific code 202 in step 152.

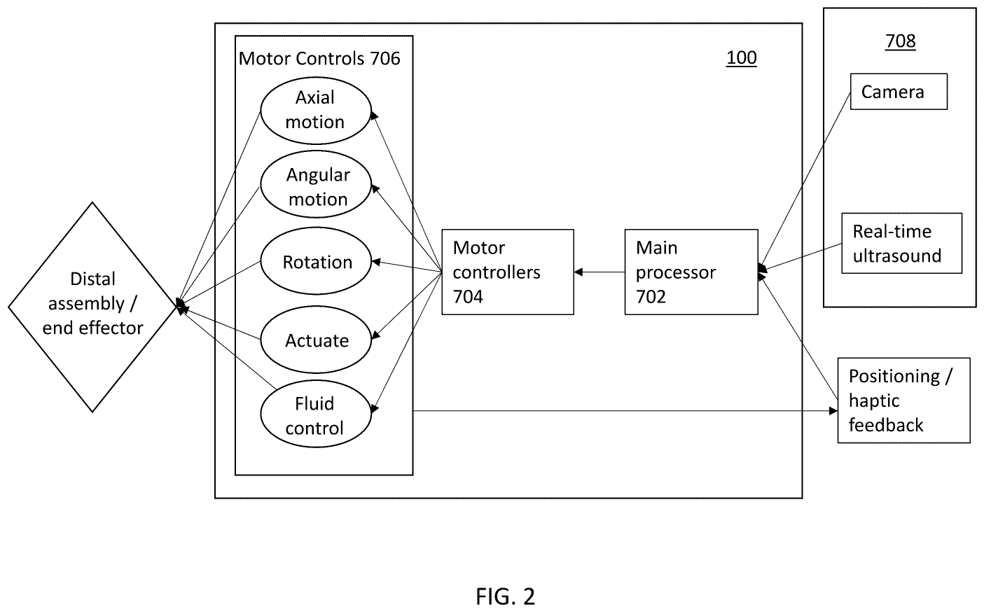

Referring now to FIG. 2, a block diagram of the universal surgical robotic platform 100 discussed with respect to FIGS. 1A and 1B is depicted. The universal surgical robotic platform 100 includes a main processor 702 which can receive the procedure-specific code 202, such as algorithms for carrying out a particular procedure, and execute the procedure-specific code 202. In accordance with the procedure-specific code, the main processor 702 can directly operate the customizable hardware components of the universal surgical robotic platform 100, such as the robotic arms 120 and adapter 130 coupled to the robotic arms 120 (and by extension the procedure-specific end effectors 300, which are coupled to the robotic arms 120 via the customized holder 400 and/or adapter 130 for instance). The universal surgical robotic platform 100 can generally include one or more motor controllers 704. The one or more motor controllers 704 can be controlled by the main processor 702 in accordance with the procedure-specific code 202 and in accordance with inputs (e.g., instructions) the main processor 702 receives from a surgeon. The one or more motor controllers 704 can impart motion to one or more customizable hardware components of the universal surgical robotic platform 100, such as robotic arms 120. The one or more robotic arms 120 can be designed and controlled to have any desirable axial, angular, or rotational motion. In some embodiments, the universal surgical robotic platform 100 can include a fluid source that can be operated to supply fluid to the procedure-specific end effectors 300, as needed.

As discussed above, the distal assembly or procedure-specific end effector 300 can be coupled to the robotic arms 120 (via the adapter 130, for instance) or other portion of the universal surgical platform 100. Through the coupling or attachment of the procedure-specific end effector 300 to the universal surgical platform 100, and for instance to robotic arms 120, the main processor 702 can operate the motor controllers 704 to move or control the procedure-specific end effector 300 in any desirable axial, angular, or rotational motion. The universal surgical robotic platform 100 (including, for instance, the robotic arms 120 or adapter 130) can also include one or more actuators, controls, or electrical connections to actuate or change the settings of the procedure-specific end effectors 300. For instance, if the procedure-specific end effectors 300 include an element for tissue ablation, the universal surgical robotic platform 100 can be connected with the procedure-specific end effectors 300 such that the universal surgical robotic platform 100 can selectively change settings of the element (e.g. ablation temperature) and control or energize the element for tissue ablation during the procedure. Ablation should be appreciated merely as an example, and the main processor 702 of the universal surgical robotic platform 100 can also provide control signals to activate a laser of the procedure-specific end effectors 300, for instance. As further examples, the procedure-specific end effectors 300 can be controlled to retract or insert a sheath or device, perform implant deployment sequences, or perform automated or semi-automated resection of specific tissue such as a prostatic lobe.

In summary of the above, the main processor 702 can control the motor controllers 704 to operate the customizable hardware components, such as robotics arms 120 or adapter 130, and therefore the procedure-specific end effectors 300 coupled to the customizable hardware components. For instance, the main processor 702, through the motor controllers 704 and robotic arms 120, can selectively position the procedure-specific end effectors 300 in the anatomy, change a setting of the procedure-specific end effectors 300 (e.g., change a laser power), and actuate the procedure-specific end effectors 300 (e.g., fire a laser).

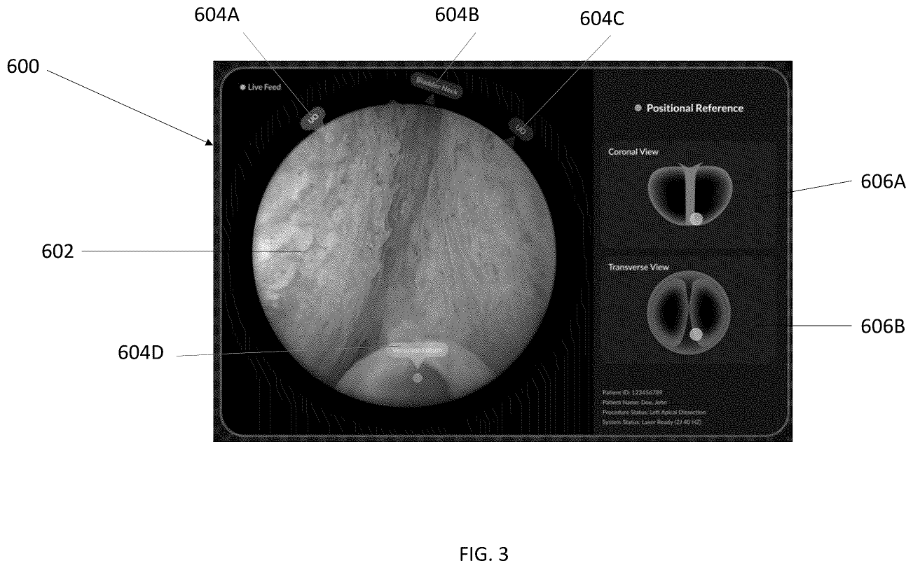

Still referring to FIG. 2, the main processor 702 can be coupled to one or more imaging devices 708, such as a camera or ultrasound device. The main processor 702 can receive and analyze imaging data in real-time, and based on the analysis, can control the motor controllers 704, robotic arms 120, and procedure-specific end effectors 300 to perform the surgical procedure as desired. The imaging data can be other data 204 discussed with respect to FIG. 1A. Merely as an example, based on the imaging data, the main processor 702 can control the motor controllers 704 to guide the procedure-specific end effectors 300 to a desired anatomical region in the body. Based on the imaging data, the main processor 702 can identify a tissue of a particular type (e.g., a lesion) and control the motor controllers 704 to operate the procedure-specific end effector 300 to treat the identified tissue (e.g., ablate the lesion). In some embodiments, the main processor 702 can analyze the imaging data to generate the GUI 600 discussed with respect to FIG. 3. In addition to or instead of the imaging data, the main processor 702 can receive feedback from one or more sensors (e.g., pressure sensors, position sensors, etc.) that are positioned on the universal surgical robotic platform 100 (such as on the robotic arms 120) and/or on the procedure-specific end effectors 300 and control operation of the robotic arms 120 and end effectors 300, as described with the imaging data, based on the feedback. The one or more sensors can provide information on procedure-specific end effector 300 position. Such information can, also, be deduced from inverse kinematics and/or motor states of the robotic platform components. Therefore, in addition to the procedure-specific code 202 that directs the operation of the universal surgical robotic platform 100 during a procedure, operation of the universal surgical robotic platform 100 can be continuously modified in real-time based on one or more feedback loops that provide real-time image data, sensor data, and/or deduced data to the main processor 702, where the real-time data relates to a current position or operation of the universal surgical robotic platform 100. It should also be appreciated that any of the other data 204 discussed above (including imaging data, inverse kinematics data, and the like) can be presented to a user (e.g. on the GUI 600), and based on the user's analysis of such data, the user can provide the universal surgical robotic platform 100 with instructions for performing the procedure.

The universal surgical robotic platform 100 can be modified or customized with the procedure-specific code 202 and procedure-specific end effectors 300 for any desirable surgical procedure involving any anatomy. For instance, the universal surgical robotic platform 100 can be used in urology. Specifically, and merely as examples, the universal surgical robotic platform 100 can be used for holmium laser enucleation of enlarged prostates, laser-based prostate-cancer focal therapy, prostate biopsy, bladder tumor resection, lithotripsy or stone removal using laser, shockwave, ultrasound, mechanical extraction devices, or other modality, and/or any other urological procedure. The universal surgical robotic platform 100 can be used to perform any desired specific procedure relating to general fields or anatomical areas, such as gynecology, the gastrointestinal tract, cardiology, and/or pulmonology. This should be appreciated as an incomplete list of examples, and the universal surgical robotic platform 100 can be customized to perform any specific procedure relating to any field or general anatomical area.

Other non-limiting examples of procedures that the universal surgical robotic platform 100 can be used to perform include, photoselective vaporization of the prostate (PVP), transurethral resection of the prostate (TURP), transperineal or transrectal prostate biopsy, transurethral resection of bladder tumor (TURBT), deployment of prostatic or urethral stent, deployment of prostatic lift device, anterior commissurotomy e.g., with a balloon, injection of botulinum toxin (Botox) to treat bladder disease, diagnostic cystoscopy or hysteroscopy which may be steered and/or aided by ML or other artificial intelligence, ablation of uterine fibroids, removal of uterine polyps, hysteroscopic myomectomy, and endometrial ablation.

As non-limiting examples outside of urology and gynecology, the universal surgical robotic platform 100 can be used for lung valve or stent placement, bronchoscopic placement of other devices for treatment of COPD, removal of foreign bodies in the lung, bronchoscopic tumor or obstruction removal, colonoscopy, polypectomy in the colon, endoscopic mucosal or submucosal resection (EMR or ESR) in the colon, stent placement in the bowel, foreign body removal from the bowel, fecal disimpaction, bowel stricture dilation, functional endoscopic sinus surgery (FESS), endoscopic septoplasty, and/or endoscopic ear surgery.

Referring now to FIGS. 1A and 3, in some embodiments, the universal surgical robotic platform 100 can include a guidance system informed by imaging (from any of the above-described imaging devices, for instance), physical position of the procedure-specific end effector 300 (discussed further below), and/or algorithms. In some embodiments, the algorithms incorporate machine learning (ML). While ML is specifically discussed herein, it should be appreciated that this is merely an example, and that the guidance systems can be informed by algorithms incorporating any artificial intelligence systems (such as neural networks or deep learning, for example) in addition to or instead of ML. In some embodiments, the guidance system can include an anatomical “global positioning system (GPS)” that informs the surgeon of the precise real-time position of the procedure-specific end effector 300 being used and the relative position of the procedure-specific end effector 300 in relation to the patient's body, organs, or certain anatomical structures.

Referring to FIG. 3, an example graphical user interface (GUI) 600 presented to a user or surgeon by the guidance system is depicted. The GUI 600 can provide a real-time feed of imaging data 602. “Landmark identification” can be informed by surgeon input, computer vision, imaging systems, and/or ML that can aid in determination of the position of anatomical structures in the patient's anatomy. The GUI 600 can present labels 640A-D over the anatomical landmarks to the user. In some embodiments, the labels 604A-D can be automatically generated based on computer vision, imaging systems, and/or ML, for instance. In some embodiments, the labels 604A-D can be added by input from the surgeon via the GUI 600. In some embodiments, the GUI 600 can present cross-sectional schematics 606A, 606B of the surgical site showing the position of the procedure-specific end effector 300 in the surgical site.

In some embodiments, the guidance system can use surgeon input, computer vision, and/or ML to identify the current phase of the procedure and/or specific surgical actions within that phase or within the entire procedure. In some cases, the system can use information about the phase, ongoing surgical actions, current procedure-specific end effector 300 position, and/or position of anatomical landmarks to alert the surgeon to possible hazards, recommend a course of action, or display additional relevant information about the surgical procedure via the GUI 600. In the specific case of holmium laser enucleation of the prostate (HoLEP) or other prostate enucleation procedures, the phases may include procedure setup, enucleation, morcellation, hemostasis, and transitional phases before, after or between those phases. Landmarks can include the bladder neck, external urethral sphincter (EUS), ureteral orifices (UOs), verumontanum, ejaculatory ducts, bladder neck fibers, prostatic blood vessels, prostatic capsule, surgical planes within the prostate and especially planes between the adenoma and capsule, stones, tumors, diverticuli, and other structures. The GPS can provide a map of the prostate, bladder, urethra, and/or other pelvic structures. The GPS can draw information from surgeon input, ML models trained on other patients, cystoscopy, ultrasound, MRI, CT, or other imaging, knowledge of the general shape and size of prostates, and other sources. In some embodiments, the guidance system can provide artificial intelligence based localization and navigation during a procedure.

With particular reference to FIGS. 4A and 4B, the detailed structure of the adapter 130 of the universal surgical robotic platform 100 and the customized holder 400 will be discussed. In some embodiments, the adapter 130 includes a body 132 and a cantilever 134 extending from the body 132. In some embodiments, the body 132 of the adapter 130 can be permanently coupled to the robotic arm 120. In some embodiments, the body 132 of the adapter 130 can be non-fixedly coupled to the robotic arm 120. In some embodiments, the cantilever 134 extends distally from the body 132 of the adapter 130. In some embodiments, the cantilever 134 extends distally from a lateral edge of the adapter 130. In some embodiments, the cantilever 134 includes a mating surface 136 at which the adapter 130 couples to the customized holder 400. In some embodiments, the mating surface 136 can be a side face of the cantilever 134. That is, in some embodiments, the mating surface 136 can be a lateral surface of the cantilever 134.

In some embodiments, the mating surface 136 can include a magnet 138. In some embodiments, the magnet 138 can be an electromagnet. While not depicted, the adapter 130 can include one or more electrical connections that provide electricity to the electromagnet 138. The one or more electrical connections can extend through the cantilever 134 or the body 132 of the adapter 130. The one or more electrical connections can be electrically coupled to a power source. In some embodiments, the power source can be provided by the universal surgical robotic platform 100. In some embodiments, the mating surface 136 of the adapter can include a plurality of protuberances 140. The plurality of protuberances 140 can extend outwardly from the mating surface 136. In some embodiments, the plurality of protuberances 140 surround the magnet 138. In some embodiments, one or more recesses 142 are defined between the plurality of protuberances 140.

In some embodiments, the adapter 130 can be configured to couple any desirable procedure-specific end effector 300 to the universal surgical robotic platform 100. More specifically, in some embodiments, a single adapter 130 can be used to couple any desirable procedure-specific end effector 300 to the universal surgical robotic platform 100. That is, a user can remove a first procedure-specific end effector 300 from the universal surgical robotic platform 100 and attach a second procedure-specific end effector 300 to the universal surgical robotic platform 100 without needing to change the adapter 130 of the universal surgical robotic platform 100.

In some embodiments, the customized holder 400 includes a base 402. The base 402 can include a mating surface 404 at which the customized holder 400 couples to the adapter 130. In some embodiments, the mating surface 404 can be a lateral surface of the base 402. In some embodiments, the mating surface 404 of the base 402 can engage the mating surface 136 of the adapter 130 to couple the customized holder 400 to the adapter 130. In some embodiments, the mating surface 404 can include a magnetic metal (not depicted). In some embodiments, the magnetic metal can be a ferrous metal. In some embodiments, the magnetic metal of the mating surface 404 can be magnetically attracted to the magnet 138. In some embodiments, the magnetic metal of the mating surface 404 of the base 402 can engage the magnet 138 of the adapter 130 to magnetically couple the adapter 130 and the customized holder 400. While the customized holder 400 is particularly described as including a magnetic metal for coupling to the magnet 138 of the adapter 130, this should be appreciated as a non-limiting example. In some embodiments, the mating surface 404 can include any material exhibiting magnetic properties such that it can magnetically couple with the magnet 138 of the adapter 130.

In some embodiments, the mating surface 404 can include a plurality of keys 406. In some embodiments, the plurality of keys 406 can surround the magnetic metal of the customized holder 400. In some embodiments, each of the plurality of keys 406 can be shaped and sized to be received in a respective recess 142 of the adapter 130. In being positioned in a recess 142, each of the keys 406 can be positioned between two of the protuberances 140. By inserting the keys 406 into a respective recess 142, a user can ensure that the customized holder 400 is coupled to the adapter 130 in a desired orientation. Engagement of the keys 406 with the respective adjacent protuberances 140 of each key can prevent the customized holder 400 from rotating out of proper orientation with the adapter 130. In some embodiments, the keys 406 can be positioned around the periphery of the mating surface 404.

In some embodiments, the customized holder 400 can include a mold 408. The mold 408 can extend from the base 402 of the customized holder 400. The mold 408 can extend from a surface of the base 402 opposite the mating surface 404. In some embodiments, the mold 408 can be shaped and sized to receive a particular procedure-specific end effector 300. In particular, in some embodiments, the mold 408 can have a shape and size that corresponds to a shape and size of the procedure-specific end effector 300. The mold 408 can be shaped and sized such that it at least partially defines a negative space corresponding to the contours of the procedure-specific end effector 300 to be received in the customized holder 400.

In some embodiments, the customized holder 400 can include a plurality of cantilevers 410. In some embodiments, the plurality of cantilevers 410 can be a pair of cantilevers 410. The plurality of cantilevers 410 can extend from the base 402 of the customized holder 400. The plurality of cantilevers 410 can extend from a surface of the base 402 opposite the mating surface 404. In some embodiments, the mold 408 can be positioned between the pair of cantilevers 410. In some embodiments, the plurality of cantilevers 410 can be positioned toward a first end of the mold 408. In some embodiments, the cantilevers 410 can define a bracket for receiving a pin therebetween, such that the cantilevers 410 define a pin joint. In some embodiments, each of the cantilevers 410 can be shaped and sized to receive a particular procedure-specific end effector 300. In particular, in some embodiments, each of the cantilevers 410 can have a shape and size that corresponds to a shape and size of the procedure-specific end effector 300. Each of the cantilevers 410 can be shaped and sized such that it at least partially defines a negative space corresponding to the contours of the procedure-specific end effector 300 to be received in the customized holder 400.

In some embodiments, the customized holder 400 can include a locking member 412. In some embodiments, the locking member 412 can be rotatably coupled to the cantilevers 410. Specifically, in some embodiments, the locking member 412 can be rotatably coupled to the cantilevers 410 at a first end of the locking member 412. In some embodiments, the locking member 412 is positioned between the pair of cantilevers 410. In some embodiments, the locking member 412 can be shaped and sized to receive a particular procedure-specific end effector 300. In particular, in some embodiments, the locking member 412 can have a shape and size that corresponds to a shape and size of the procedure-specific end effector 300. The locking member 412 can be shaped and sized such that it at least partially defines a negative space corresponding to the contours of the procedure-specific end effector 300 to be received in the customized holder 400.

In some embodiments, the customized holder 400 can include a locking member 412. In some embodiments, the locking member 412 can be rotatably coupled to the cantilevers 410. Specifically, in some embodiments, the locking member 412 can be rotatably coupled to the cantilevers 410 at a first end of the locking member 412. In some embodiments, the locking member 412 is positioned between the pair of cantilevers 410. In some embodiments, the locking member 412 can be shaped and sized to receive a particular procedure-specific end effector 300. In particular, in some embodiments, the locking member 412 can have a shape and size that corresponds to a shape and size of the procedure-specific end effector 300. The locking member 412 can be shaped and sized such that it at least partially defines a negative space corresponding to the contours of the procedure-specific end effector 300 to be received in the customized holder 400.

In some embodiments, the customized holder 400 can include a retainer 414. In some embodiments, the retainer 414 can be positioned at a second end of the mold 408 opposite the cantilevers 410. In some embodiments, the retainer 414 can be configured to receive and retain the second, free end of the locking member 412. In some embodiments, the retainer 414 can include a threaded member to be received in the second, free end of the locking member 412 and a nut, or other threaded fastener, can be secured to the threaded member to hold the locking member 412 in a closed position. It should be appreciated that the retainer 414 can include any desirable fastening mechanism to receive and hold the second, free end of the locking member 412. For instance, in some embodiments, the second, free end of the locking member 412 and the retainer 414 can include corresponding features such that the locking member 412 and the retainer 414 can engage each other in a snap-fit arrangement. As another example, in some embodiments, the second, free end of the locking member 412 and the retainer 414 can include correspondingly dimensioned features such that the locking member 412 and the retainer 414 can engage each other in a friction-fit arrangement. As another example, in some embodiments, the second, free end of the locking member 412 and the retainer 414 can include corresponding features such that the locking member 412 and the retainer 414 can magnetically couple.

The retainer 414 can be configured to hold the locking member 412 in a closed position. When the locking member 412 is in the closed position, as shown in FIG. 4A, the locking member 412, the cantilevers 410, and the mold 408 can define a negative space shaped and sized for securing a specific procedure-specific end effector 300 in the customized holder 400. For instance, the internal surfaces (e.g., surfaces defining the negative space) of the locking member 412, the cantilevers 410, and the mold 408 can be contoured, shaped, and sized such that they define a negative space corresponding to a circumference of the procedure-specific end effector 300 at a position along the procedure-specific end effector 300 to be received in the customized holder 400. The shape and size of the locking member 412, the cantilevers 410, and the mold 408 can ensure that the particular procedure-specific end effector 300 is retained in the customized holder 400 during a procedure, for instance, without allowing unwanted movement of the procedure-specific end effector 300 relative the customized holder 400. The shape and size of the locking member 412, the cantilevers 410, and the mold 408 can ensure that the functionality (e.g., movement and actuation) of the particular procedure-specific end effector 300 is not inhibited by the customized holder 400.

A plurality of customized holders 400 can be used with the universal surgical robotic platform 100. Each of the plurality of customized holders 400 can include the same base 402 and mating surface 404. Therefore, each of the plurality of customized holders 400 can couple with the mating surface 136 of a single adapter 130 of the universal surgical robotic platform 100. Each of the plurality of customized holders can include uniquely shaped and sized locking members 412, cantilevers 410, and molds 408 such that each of the customized holders 400 is particularly shaped and sized for receiving and retaining a single, particular procedure-specific end effector 300.

Therefore, merely as an example, a user can select a first procedure-specific end effector 300 for use during a first procedure. The user can then select a first customized holder 400 that is shaped and sized particularly for holding and retaining the first procedure-specific end effector 300. The first procedure-specific end effector 300 can then be placed in the first customized holder 400, and the locking member 412 of the first customized holder 400 can be brought to the closed position to retain the first procedure-specific end effector 300 in the customized holder 400. The first customized holder 400 can then be coupled to the adapter 130 of the universal surgical robotic platform 100 for performing the first procedure. It should be appreciated that, in some embodiments, the first customized holder 400 can first be coupled to the adapter 130 and the first procedure-specific end effector 300 can then be placed in the customized holder 400. After completion of the first procedure, a user can remove the first customized holder 400, including the first procedure-specific end effector 300, from the universal surgical robotic platform 100. A user can then select a second procedure-specific end effector 300 for use during a second procedure. The user can then select a second customized holder 400 that is shaped and sized particularly for holding and retaining the second procedure-specific end effector 300. The second procedure-specific end effector 300 can then be placed in second first customized holder 400, and the locking member 412 of the second customized holder 400 can be brought to the closed position to retain the second procedure-specific end effector 300 in the customized holder 400. The second customized holder 400 can then be coupled to the adapter 130 of the universal surgical robotic platform 100 for performing the second procedure. Accordingly, different procedure-specific end effectors 300 can be used with the universal surgical robotic platform 100 without modifying the universal surgical robotic platform.

In some embodiments, where the magnet 138 of the adapter 130 is an electromagnet, the adapter 130 and the customized holder 400 can be configured to decouple in the case of a loss of power during a procedure. Particularly, a loss of current to the electromagnet 138 can cause the electromagnet 138 to decouple from the magnet of the customized holder 400. Doing so can prevent injury to a patient that the universal surgical robotic platform 100 is being used to operate on. Particularly, from decoupling the customized holder 400 (which can be holding the procedure-specific end effector 300 in the anatomy of the user) from the adapter 130, it can be ensured that the weight of the robotic arm 120 is not applied to the anatomy of the user (e.g., when the robotic arm loses motor control), which can prevent injury to the patient.

In some embodiments, any or all of the size or strength of the magnet 138, the size or density of the magnetic material of the mating surface 404 of the customized holder 400, the size and shape of the protuberances 140, the size and shape of the recesses 142, and the size and shape of the keys 406 can be particularly selected to achieve a desired strength of coupling between the adapter 130 and the customized holder 400. Particularly, the desired strength of coupling can allow for a desired torque-effected decoupling of the customized holder 400 from the adapter 130 if undesirably large torques are imparted on the procedure-specific end effector 300 (and therefore the junction between the adapter 130 and the customized holder 400 due to the various couplings).

It should be appreciated from the above description that the universal surgical robotic platform 100 can be modified to perform any type of desired procedure throughout the lifetime of the universal surgical robotic platform 100. That is, the universal surgical robotic platform 100 can be continually modified with the necessary procedure-specific code 202 and procedure-specific end effectors 300 to perform different procedures, as desired. For instance, a user can upload a procedure-specific code 202 for a first type of procedure to the universal surgical robotic platform 100 and attach one or more procedure-specific end effectors 300 for the first type of procedure to the universal surgical robotic platform 100. The universal surgical robotic platform 100 can then carry out the first type of procedure. Following completion of the first procedure, the user can remove the procedure-specific end effector 300 for the first type of procedure from the universal surgical robotic platform 100. The user can then upload a procedure-specific code 202 for a second type of procedure to the universal surgical robotic platform 100 and attach one or more procedure-specific end effectors 300 for the second type of procedure to the universal surgical robotic platform 100, and the universal surgical robotic platform 100 can then carry out the second type of procedure.

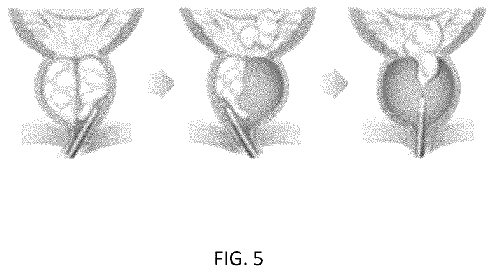

Referring to FIG. 5, the universal surgical robotic platform 100 can significantly improve HoLEP procedures. Procedure-specific code 202 can be uploaded to the universal surgical robotic platform 100 to perform HoLEP or variant procedures (e.g., ejaculation-sparing HoLEP, thulium laser enucleation of the prostate (ThuLEP), greenlight laser enucleation of the prostate (GreenLEP)). Specific to HoLEP, procedure-specific end effectors 300, such as a rigid cystoscope, resectoscope, or nephroscope, a cystoscopy, resectoscopy, or nephroscopy sheath, a holmium, thulium (for ThuLEP), greenlight (for GreenLEP), or blue light laser, a laser fiber, a morcellator, and/or an irrigation system can be coupled to customizable hardware components, such as robotic arms 120 or adapter 130, of the universal surgical robotic platform 100. During HoLEP procedures, the universal surgical robotic platform 100 can control the robotic arms 120 to position the procedure-specific end effectors 300 at the prostate of a patient (with or without guidance from real-time feedback data, as described above), and the universal surgical robotic platform 100 can control the procedure-specific end effectors 300 to fire a laser, activate a morcellator, manipulate prostatic tissue, and/or modify a laser power in order to remove adenoma from the prostate.

The universal surgical robotic platform 100 can perform surgical procedures fully autonomously by means of the procedure-specific code 202, and optionally, the above-discussed feedback data. The universal surgical robotic platform 100 can also be operated manually or semi-autonomously. In such cases, the real-time feedback data (e.g., imaging data or other sensor data) can be utilized by a human operator to control or adjust operation of the universal surgical robotic platform 100. In such cases, the universal surgical robotic platform 100 can provide one or more alerts or notifications, such as haptic feedback, to a user based on the feedback data. The universal surgical robotic platform 100 can be used to perform any desired endoscopic procedure.

While embodiments have been discussed utilizing real-time imaging data to guide the universal surgical robotic platform 100 or assist a user in controlling operation of the universal surgical robotic platform 100, it should be appreciated that imaging data captured pre-operatively can also be used to guide or control operation of the universal surgical robotic platform 100.

Techniques operating according to the principles described herein may be implemented in any suitable manner. The above-described processing steps and acts can be included in algorithms that carry out the various processes discussed above. Algorithms derived from these processes may be implemented as software integrated with and directing the operation of one or more single- or multi-purpose processors, may be implemented as functionally-equivalent circuits such as a Digital Signal Processing (DSP) circuit, Field Programmable Gate Array (FPGA), or an Application-Specific Integrated Circuit (ASIC), or may be implemented in any other suitable manner.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. For instance, the program-specific code discussed herein can include one or more functional facilities for carrying out the above-described methods. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

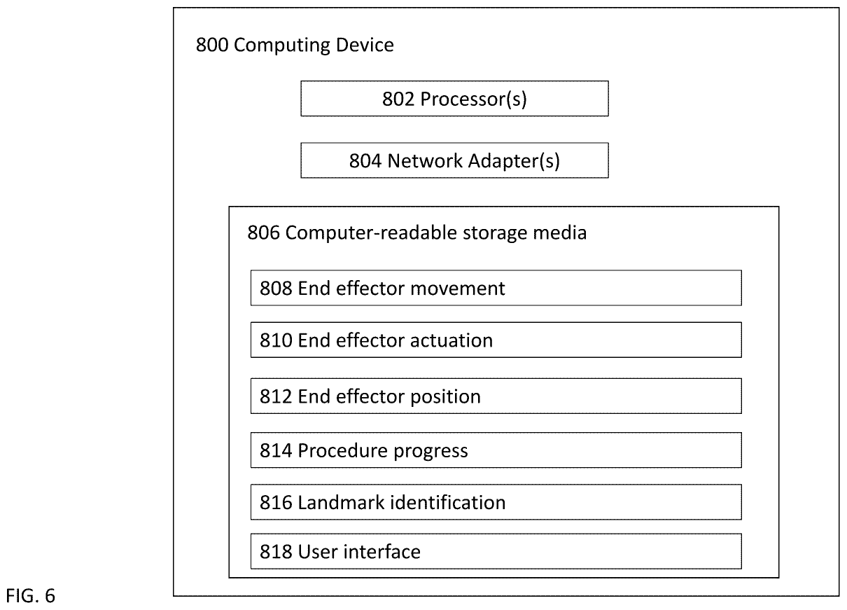

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 806 of FIG. 6 described below (i.e., as a portion of a computing device 800) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 6 illustrates one exemplary implementation of a computing device in the form of a computing device 800 that may be used in a system implementing the techniques described herein, although others are possible. It should be appreciated that FIG. 6 is intended neither to be a depiction of necessary components for a computing device to operate in accordance with the principles described herein, nor a comprehensive depiction.

Computing device 800 may comprise at least one processor 802 (e.g., the processor 702 discussed with respect to FIG. 2), a network adapter 804, and computer-readable storage media 806, which can store both procedure-specific code and universal code (e.g., code not particular to carrying out a specific procedure). Computing device 800 may be, for example, a desktop or laptop personal computer, a personal digital assistant (PDA), a smart mobile phone, a server, a wireless access point or other networking element, an on board component of the universal surgical robotic platform 100, or any other suitable computing device. Network adapter 804 may be any suitable hardware and/or software to enable the computing device 800 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 806 may be adapted to store data to be processed and/or instructions to be executed by one or more processors 802. Processor 802 enables processing of data and execution of instructions. The data and instructions may be stored on the computer-readable storage media 806.

The data and instructions stored on computer-readable storage media 806 may comprise computer-executable instructions implementing techniques which operate according to the principles described herein. In the example of FIG. 6, computer-readable storage media 806 stores computer-executable instructions implementing various facilities and storing various information as described above. Computer-readable storage media 806 may store an end effector movement facility 808, which when executed by the processor 802, enables to the processor 802 to move the end effector 300 (based on feedback data and/or user instructions for instance). Computer-readable storage media 806 may store an end effector actuation facility 810, which when executed by the processor 802, enables the processor 802 to actuate the effector 300 (based on feedback data and/or user instructions for instance). Computer-readable storage media 806 may store an end effector position facility 812, which when executed by the processor 802, enables the processor 802 to determine the position of the effector 300 (based on feedback data such as imaging or motor control data). Computer-readable storage media 806 may store a procedure progress module 814, which when executed by the processor 802, enables the processor 802 to determine a progress of a first procedure (based on end effector 300 position and actuation, for instance). Computer-readable storage media 806 may store a landmark identification facility 816, which when executed by the processor 802, enables the processor 802 to identify relevant anatomical landmarks in received imaging data. Computer-readable storage media 806 may store a user interface facility 818, which when executed by the processor 802, enables the processor 802 to generate a graphical user interface 600 for the user.

While not illustrated in FIG. 6, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format

Based on the foregoing, it should now be understood that embodiments shown and described herein relate to universal surgical robotic platform.

Non-limiting embodiments of the present disclosure are set out in the following clauses:

Clause 1. A robotic system, comprising: a processor; and a robotic platform in communication with the processor, wherein: the robotic platform is configured to couple to a first procedure-specific end effector, the first procedure-specific end effector controllable by the robotic platform and being selected to perform a first type of procedure; and the processor is configured to receive first procedure-specific code and to execute the first procedure-specific code to cause the robotic platform to operate the first procedure-specific end effector to perform the first type of procedure.

Clause 2. The robotic system of clause 1, wherein: the robotic platform comprises one or more robotic arms; and the one or more robotic arms are configured to be coupled to the first procedure-specific end effector.

Clause 3. The robotic system of clause 1 or clause 2, wherein the processor is configured to: receive imaging data from one or more imaging devices; analyze the imaging data; and present a graphical user interface of a surgical site based on the imaging data.

Clause 4. The robotic system of any one of clauses 1-3, wherein the processor is configured to, via the graphical user interface, inform a user of a real-time position of the first procedure-specific end effector in a surgical site.

Clause 5. The robotic system of any one of clauses 1-4, wherein the processor is configured to: identify one or more anatomical landmarks in the imaging data; and label the one or more anatomical landmarks in the imaging data on the graphical user interface.

Clause 6. The robotic system of any one of clauses 1-5, wherein the processor is configured to: receive imaging data from one or more imaging devices; analyze the imaging data; and control the first procedure-specific end effector based on the imaging data.

Clause 7. The robotic system of any one of clauses 1-6, wherein: the robotic platform is configured to couple to a second procedure-specific end effector, the second procedure-specific end effector controllable by the robotic platform and being selected to perform a second type of procedure; and the processor is configured to receive second procedure-specific code and to execute the second procedure-specific code to cause the robotic platform to operate the second procedure-specific end effector to perform the second type of procedure.

Clause 8. The robotic system of any one of clauses 1-7, wherein: the robotic platform comprises an adapter positioned at a distal end of one or more robotic arms of the robotic platform; and the robotic platform is configured to couple to the first procedure-specific end effector via the adapter.

Clause 9. The robotic system of any one of clauses 1-8, wherein the adapter is configured to couple to a customized holder configured to hold the first procedure-specific end effector.

Clause 10. The robotic system of any one of clauses 1-9, wherein the customized holder is shaped and sized based on the first procedure-specific end effector.

Clause 11. A method, comprising: uploading a first procedure-specific code to a robotic platform, wherein the first procedure-specific code, when executed by a processor of the robotic platform, causes the robotic platform to operate a first procedure-specific end effector to perform a first type of procedure; attaching the first procedure-specific end effector to the robotic platform; and causing the robotic platform to perform the first type of procedure.

Clause 12. The method of clause 11, further comprising: uploading a second procedure-specific code to the robotic platform, wherein the second procedure-specific code, when executed by the processor of the robotic platform, causes the robotic platform to operate the first procedure-specific end effector to perform a second type of procedure; and causing the robotic platform to perform the second type of procedure.

Clause 13. The method of clause 11 or clause 12, wherein attaching the first procedure-specific end effector to the robotic platform comprises attaching the first procedure-specific end effector to the robotic platform via a first customized holder, wherein the first customized holder is shaped and sized based on the first procedure-specific end effector.

Clause 14. The method of any one of clauses 11-13, further comprising: removing the first procedure-specific end effector from the robotic platform; uploading a second procedure-specific code to the robotic platform, wherein the second procedure-specific code, when executed by the processor of the robotic platform, causes the robotic platform to operate a second procedure-specific end effector to perform a second type of procedure; attaching the second procedure-specific end effector to the robotic platform; and causing the robotic platform to perform the second type of procedure.

Clause 15. The method of any one of clauses 11-14, wherein: attaching the first procedure-specific end effector to the robotic platform comprises attaching the first procedure-specific end effector to the robotic platform via a first customized holder, wherein the first customized holder is shaped and sized based on the first procedure-specific end effector; and attaching the second procedure-specific end effector to the robotic platform comprises attaching the second procedure-specific end effector to the robotic platform via a second customized holder, wherein the second customized holder is shaped and sized based on the second procedure-specific end effector.

Clause 16. The method of any one of clauses 11-15, further comprising: removing the first procedure-specific end effector and the first customized holder from the robotic platform prior to attaching the second procedure-specific end effector and the second customized holder to the robotic platform.

Clause 17. A system for coupling an end effector to a robotic platform, comprising: an adapter configured to be coupled to a robotic arm of the robotic platform; and a customized holder couplable to the adapter, wherein the customized holder is configured to hold the end effector.

Clause 18. The system of clause 17, wherein the adapter and the customized holder are magnetically couplable.

Clause 19. The system of clause 17 or clause 18, wherein: the adapter comprises an electromagnet; the customized holder comprises a magnetic material; and the adapter and the customized holder are configured to magnetically couple via the electromagnet of the adapter and the magnetic material of the customized holder.

Clause 20. The system of any one of clauses 17-19, wherein the adapter comprises: a body configured to be coupled to the robotic arm; and a cantilever extending distally from the body, wherein the cantilever comprises a side face defining a mating surface of the adapter at which the adapter is configured to couple to the customized holder.

Clause 21. The system of any one of clauses 17-20, wherein the mating surface of the adapter comprises an electromagnet.

Clause 22. The system of any one of clauses 17-21, wherein the mating surface of the adapter comprises a plurality of protuberances defining one or more recesses therebetween.

Clause 23. The system of any one of clauses 17-22, wherein: the mating surface of the adapter comprises an electromagnet; and the plurality of protuberances surround the electromagnet.

Clause 24. The system of any one of clauses 17-23, wherein the customized holder comprises a mating surface of the customized holder at which the customized holder is configured to couple to the mating surface of the adapter.

Clause 25. The system of any one of clauses 17-24, wherein the mating surface of the customized holder comprises a plurality of keys configured to be received in the one or more recesses.

Clause 26. The system of any one of clauses 17-25, wherein the customized holder comprises: a base defining a mating surface of the customized holder at which the customized holder is couplable to the adapter; a plurality of cantilevers extending from the base; a locking member rotatably coupled to the plurality of cantilevers at a first end of the locking member; and a retainer configured to receive a second end of the locking member.

Clause 27. The system of any one of clauses 17-26, wherein the customized holder is configured to hold the end effector in a space defined at least partially between the plurality of cantilevers and the locking member when the second end of the locking member is received by the retainer.

Clause 28. The system of any one of clauses 17-27, wherein: the plurality of cantilevers are shaped and sized based on the end effector to be held in the space defined at least partially between the plurality of cantilevers and the locking member when the second end of the locking member is received by the retainer; and the locking member is shaped and sized based on the end effector to be held in the space defined at least partially between the plurality of cantilevers and the locking member when the second end of the locking member is received by the retainer.

Clause 29. An adapter, comprising: a body comprising: a first end couplable to a robotic arm; and a second end; and a cantilever extending distally from the second end of the body, wherein the cantilever comprises a side face defining a mating surface of the adapter at which the adapter is configured to couple to a customized holder, wherein the mating surface comprises: an electromagnet; and a plurality of protuberances surrounding the electromagnet and defining one or more recesses between the plurality of protuberances.

Clause 30. A customized holder, comprising: a base defining a mating surface at which the customized holder is configured to couple to a mating surface of an adapter, wherein the mating surface comprises: a magnetic metal; and a plurality of keys surrounding the magnetic metal; a plurality of cantilevers extending from the base; a locking member rotatably coupled to the plurality of cantilevers at a first end of the locking member; and a retainer configured to receive a second end of the locking member, wherein the customized holder is configured to hold an end effector in a space defined at least partially between the plurality of cantilevers and the locking member when the second end of the locking member is received by the retainer; the plurality of cantilevers are shaped and sized based on the end effector to be held in the space defined at least partially between the plurality of cantilevers and the locking member when the second end of the locking member is received by the retainer; and the locking member is shaped and sized based on the end effector to be held in the space defined at least partially between the plurality of cantilevers and the locking member when the second end of the locking member is received by the retainer.

All patents and publications mentioned in this specification are herein incorporated by reference to the same extent as if each independent patent and publication was specifically and individually indicated to be incorporated by reference.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of any appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by any appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

What is claimed is:

1. A robotic system, comprising:
a processor; and
a robotic platform in communication with the processor, wherein:
the robotic platform is configured to couple to a first procedure-specific end effector, the first procedure-specific end effector controllable by the robotic platform and being selected to perform a first type of procedure; and
the processor is configured to receive first procedure-specific code and to execute the first procedure-specific code to cause the robotic platform to operate the first procedure-specific end effector to perform the first type of procedure.

2. The robotic system of claim 1, wherein:
the robotic platform comprises one or more robotic arms; and
the one or more robotic arms are configured to be coupled to the first procedure-specific end effector.

3. The robotic system of claim 1, wherein the processor is configured to:
receive imaging data from one or more imaging devices;
analyze the imaging data; and
present a graphical user interface of a surgical site based on the imaging data.

4. The robotic system of claim 3, wherein the processor is configured to, via the graphical user interface, inform a user of a real-time position of the first procedure-specific end effector in a surgical site.

5. The robotic system of claim 3, wherein the processor is configured to:
identify one or more anatomical landmarks in the imaging data; and
label the one or more anatomical landmarks in the imaging data on the graphical user interface.

6. The robotic system of claim 1, wherein the processor is configured to:
receive imaging data from one or more imaging devices;
analyze the imaging data; and
control the first procedure-specific end effector based on the imaging data.

7. The robotic system of claim 1, wherein:
the robotic platform is configured to couple to a second procedure-specific end effector, the second procedure-specific end effector controllable by the robotic platform and being selected to perform a second type of procedure; and
the processor is configured to receive second procedure-specific code and to execute the second procedure-specific code to cause the robotic platform to operate the second procedure-specific end effector to perform the second type of procedure.

8. The robotic system of claim 1, wherein:
the robotic platform comprises an adapter positioned at a distal end of one or more robotic arms of the robotic platform; and
the robotic platform is configured to couple to the first procedure-specific end effector via the adapter.

9. The robotic system of claim 8, wherein the adapter is configured to couple to a customized holder configured to hold the first procedure-specific end effector.

10. The robotic system of claim 9, wherein the customized holder is shaped and sized based on the first procedure-specific end effector.

11. A method, comprising:
uploading a first procedure-specific code to a robotic platform, wherein the first procedure-specific code, when executed by a processor of the robotic platform, causes the robotic platform to operate a first procedure-specific end effector to perform a first type of procedure;
attaching the first procedure-specific end effector to the robotic platform; and
causing the robotic platform to perform the first type of procedure.

12. The method of claim 11, further comprising:
uploading a second procedure-specific code to the robotic platform, wherein the second procedure-specific code, when executed by the processor of the robotic platform, causes the robotic platform to operate the first procedure-specific end effector to perform a second type of procedure; and
causing the robotic platform to perform the second type of procedure.

13. The method of claim 11, wherein attaching the first procedure-specific end effector to the robotic platform comprises attaching the first procedure-specific end effector to the robotic platform via a first customized holder, wherein the first customized holder is shaped and sized based on the first procedure-specific end effector.

14. The method of claim 11, further comprising:
removing the first procedure-specific end effector from the robotic platform;
uploading a second procedure-specific code to the robotic platform, wherein the second procedure-specific code, when executed by the processor of the robotic platform, causes the robotic platform to operate a second procedure-specific end effector to perform a second type of procedure;
attaching the second procedure-specific end effector to the robotic platform; and
causing the robotic platform to perform the second type of procedure.

15. The method of claim 14, wherein:
attaching the first procedure-specific end effector to the robotic platform comprises attaching the first procedure-specific end effector to the robotic platform via a first customized holder, wherein the first customized holder is shaped and sized based on the first procedure-specific end effector; and
attaching the second procedure-specific end effector to the robotic platform comprises attaching the second procedure-specific end effector to the robotic platform via a second customized holder, wherein the second customized holder is shaped and sized based on the second procedure-specific end effector.

16. The method of claim 15, further comprising:
removing the first procedure-specific end effector and the first customized holder from the robotic platform prior to attaching the second procedure-specific end effector and the second customized holder to the robotic platform.

17. A system for coupling an end effector to a robotic platform, comprising:
an adapter configured to be coupled to a robotic arm of the robotic platform; and
a customized holder couplable to the adapter, wherein the customized holder is configured to hold the end effector.

18. The system of claim 17, wherein the adapter and the customized holder are magnetically couplable.

19. The system of claim 17, wherein:
the adapter comprises an electromagnet;
the customized holder comprises a magnetic material; and
the adapter and the customized holder are configured to magnetically couple via the electromagnet of the adapter and the magnetic material of the customized holder.

20. The system of claim 17, wherein the adapter comprises:
a body configured to be coupled to the robotic arm; and
a cantilever extending distally from the body, wherein the cantilever comprises a side face defining a mating surface of the adapter at which the adapter is configured to couple to the customized holder.

21. The system of claim 20, wherein the mating surface of the adapter comprises an electromagnet.

22. The system of claim 20, wherein the mating surface of the adapter comprises a plurality of protuberances defining one or more recesses therebetween.

23. The system of claim 22, wherein:
the mating surface of the adapter comprises an electromagnet; and
the plurality of protuberances surround the electromagnet.

24. The system of claim 22, wherein the customized holder comprises a mating surface of the customized holder at which the customized holder is configured to couple to the mating surface of the adapter.

25. The system of claim 24, wherein the mating surface of the customized holder comprises a plurality of keys configured to be received in the one or more recesses.

26. The system of claim 17, wherein the customized holder comprises:
a base defining a mating surface of the customized holder at which the customized holder is couplable to the adapter;
a plurality of cantilevers extending from the base;
a locking member rotatably coupled to the plurality of cantilevers at a first end of the locking member; and
a retainer configured to receive a second end of the locking member.

27. The system of claim 26, wherein the customized holder is configured to hold the end effector in a space defined at least partially between the plurality of cantilevers and the locking member when the second end of the locking member is received by the retainer.

28. The system of claim 27, wherein:
the plurality of cantilevers are shaped and sized based on the end effector to be held in the space defined at least partially between the plurality of cantilevers and the locking member when the second end of the locking member is received by the retainer; and
the locking member is shaped and sized based on the end effector to be held in the space defined at least partially between the plurality of cantilevers and the locking member when the second end of the locking member is received by the retainer.

29. An adapter, comprising:
a body comprising:
a first end couplable to a robotic arm; and
a second end; and
a cantilever extending distally from the second end of the body, wherein the cantilever comprises a side face defining a mating surface of the adapter at which the adapter is configured to couple to a customized holder, wherein the mating surface comprises:
an electromagnet; and
a plurality of protuberances surrounding the electromagnet and defining one or more recesses between the plurality of protuberances.

30. A customized holder, comprising:
a base defining a mating surface at which the customized holder is configured to couple to a mating surface of an adapter, wherein the mating surface comprises:
a magnetic metal; and
a plurality of keys surrounding the magnetic metal;
a plurality of cantilevers extending from the base;
a locking member rotatably coupled to the plurality of cantilevers at a first end of the locking member; and
a retainer configured to receive a second end of the locking member, wherein
the customized holder is configured to hold an end effector in a space defined at least partially between the plurality of cantilevers and the locking member when the second end of the locking member is received by the retainer;
the plurality of cantilevers are shaped and sized based on the end effector to be held in the space defined at least partially between the plurality of cantilevers and the locking member when the second end of the locking member is received by the retainer; and
the locking member is shaped and sized based on the end effector to be held in the space defined at least partially between the plurality of cantilevers and the locking member when the second end of the locking member is received by the retainer.
